(19) United States
(12) Patent Application Publication
(10) Pub. No.: US 2020/0370033 A1
(43) Pub. Date: Nov. 26, 2020

(54) BIOLOGICAL ASSEMBLY INCLUDING BIOLOGICAL COMPONENT AND SHIELD

(71) Applicant: Regents of the University of Minnesota, Minneapolis, MN (US)

(72) Inventors: Jonathan Konstantine Sakkos, Lansing, MI (US); Alptekin Aksan, Minneapolis, MN (US); Lawrence P. Wackett, St. Paul, MN (US)

(21) Appl. No.: 16/959,812

(22) PCT Filed: Dec. 31, 2018
(86) PCT No.: PCT/US18/68154

§ 371 Date: Jul. 2, 2020

Related U.S. Application Data
(60) Provisional application No. 62/613,308, filed on Jan. 3, 2018.

(51) Int. Cl. C12N 11/14; C12P 17/12; C12P 7/42

(52) U.S. Cl. CPC C12N 11/14; C12P 7/42; C12P 17/12

(57) ABSTRACT

According to various embodiments of the present disclosure, a biological assembly for performing biocatalysis includes a biological component. The assembly further includes a porous shell layer at least partially coating the biological component. The porous shell layer includes an inorganic network having a cationic component and an anionic component.

### CROSS-REFERENCE TO RELATED APPLICATION

This application claims the benefit of priority to U.S. Provisional Patent Application Ser. No. 62/613,308 entitled “BIOLOGICAL ASSEMBLY INCLUDING BIOLOGICAL COMPONENT AND SHIELD,” filed Jan. 3, 2018, the disclosure of which is incorporated herein in its entirety by reference.

### BACKGROUND

Various biological components can be used to catalyze desired reactions. However, certain biological components can lack stability when subjected to various stresses or environmental conditions. In order to use biological components more effectively it is desirable to develop systems and method for developing assemblies for biological components.

### SUMMARY OF THE DISCLOSURE

According to various embodiments of the present disclosure, a biological assembly for performing biocatalysts includes a biological component. The assembly further includes a porous shell layer at least partially coating the biological component. The porous shell layer includes an inorganic network having a cationic component and an anionic component.

According to various embodiments, a method of making a biological assembly is disclosed. The biological assembly includes a biological component. The assembly further includes a porous shell layer at least partially coating the biological component. The porous shell layer includes an inorganic network having a cationic component and an anionic component. The method includes providing or receiving the biological component. The method further includes contacting the biological component with the anionic component and the cationic component to form the porous shell layer.

According to various embodiments, a method of using a biological assembly is disclosed. The biological assembly includes a biological component. The assembly further includes a porous shell layer at least partially coating the biological component. The porous shell layer includes an inorganic network having a cationic component and an anionic component. The method includes contacting the biological component with a catalyzable reactant. The method further includes catalyzing a reaction of the catalyzable reactant to a product.

There are various non-limiting advantages to use the assemblies described herein, some of which are unexpected. For example, according to various embodiments, the biological structure in the assembly can substantially retain the ability to perform biocatalysis when the assembly is subjected to various stresses such as osmotic shock, lysozyme treatment, desiccation, freezing, thawing, heating, exposure to protozoa, exposure to bacteriophage, changes in pH, mechanical stress, fluid sheer stress, or combinations thereof. Additionally, according to various embodiments of the present disclosure, the assembly can allow the biological component to be heated to a higher temperature than a corresponding biological component that is free of the porous shell layer. According to various embodiments, heating the biological component can provide an increased reaction rate.

### BRIEF DESCRIPTION OF THE FIGURES

The drawings illustrate generally, by way of example, but not by way of limitation, various embodiments discussed in the present document.

FIGS. 1A-1B shows a diagram of a layer by layer coating process involved in creation of a microbial exoskeleton and images of the process, in accordance with various embodiments.

FIG. 2 is a graph showing apparent pore diameter measured from SEM images, n>1,300, in accordance with various embodiments.

FIG. 3 is a graph showing the effect of number of layers deposited on cell envelope permeability to 1-N-phenylnaphthylamine (NPN), and propidium iodide (PI), in accordance with various embodiments.

FIG. 4 is a graph showing the effect of the number of layers on biocatalytic activity with homoprotocatechuate 2,3-dioxygenase (HPCD), in accordance with various embodiments.

FIGS. 5A-5D is a set of graphs showing the effect of surfactant treatment (1% w/v for 20 minutes) on cell envelope permeability, in accordance with various embodiments.

FIGS. 6A-6F are a series of graphs showing protection offered by the exoskeleton against a variety of stresses, in accordance with various embodiments.

FIG. 7 is a graph showing protein retention in phosphate buffer (pH 5.8) after 5 days with daily buffer washes to remove leaked protein, in accordance with various embodiments.

FIG. 8 is a graph showing biocatalysis at elevated temperatures, in accordance with various embodiments.

### DETAILED DESCRIPTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2:2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term is used to refer to a nonexclusive “or” unless otherwise indicated. The statement “at least one of A and B” has the same meaning as “A, B, or A and B.” In addition, it is to be understood. that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

In the methods described herein, the acts can be carried out in any order without departing from the principles of the disclosure, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%.

The term “organic group” as used herein refers to any carbon-containing functional group. Examples can include an oxygen-containing group such as an alkoxy group, aryloxy group, aralkyloxy group, oxo(carbonyl) group; a carboxyl group including a carboxylic acid, carboxylate, and a carboxylate ester; a sulfur-containing group such as an alkyl and aryl sulfide group; and other heteroatom-containing groups. Non-limiting examples of organic groups include OR, OOR, OC(O)N(R)₂, CN, CF₃, OCF₃, R, C(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, C(═NOR)R, and substituted or unsubstituted (C₁-C₁₀₀)hydrocarbyl, wherein R can be hydrogen (in examples that include other carbon atoms) or a carbon-based moiety, and wherein the carbon-based moiety can be substituted or unsubstituted.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can he or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thione), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀), alkyl, acyl, cycloalkyl, aryl; or wherein two R groups bonded to a nitrogen atom.

The term “alkyl” as used herein refers to straight chain and branched alkyl groups and cycloalkyl groups having from 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkenyl” as used herein refers to straight and branched chain and cyclic alkyl groups as defined herein, except that at least one double bond exists between two carbon atoms, Thus, alkenyl groups have from 2 to about 20 carbon atoms, or 2 to 12 carbon atoms or, in some embodiments, from 2 to 8 carbon atoms. Examples include, but are not limited to vinyl, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

The term “alkynyl” as used herein refers to straight and branched chain alkyl groups, except that at least one triple bond exists between two carbon atoms. Thus, alkynyl groups have from 2 to about 20 carbon atoms, or from 2 to 12 carbons or, in some embodiments, from 2 to 8 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

The term “acyl” as used herein refers to a group containing a carbonyl moiety wherein the group is bonded via the carbonyl carbon atom. The carbonyl carbon atom is bonded to a hydrogen forming a “formyl” group or is bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. An acyl group can include 0 to about 12, 0 to about 20, or 0 to about 40 additional carbon atoms bonded to the carbonyl group. An acyl group can include double or triple bonds within the meaning herein. An acryloyl group is an example of an acyl group. An acyl group can also include heteroatoms within the meaning herein. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4- 2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

The term “aryl” as used herein refers to cyclic aromatic hydrocarbon groups that do not contain heteroatoms in the ring. Thus, aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof.

The term “room temperature” as used herein refers to a temperature of about 15° C. to 28° C.

The polymers described herein can terminate in any suitable way. In some embodiments, the polymers can terminate with an end group that is independently chosen from a suitable polymerization initiator, —H, —OH, a substituted or unsubstituted (C₁-C₂₀)hydrocarbyl (e.g., (C₁-C₁₀)alkyl or (C₆-C₂₀)aryl) interrupted with 0, 1, 2, or 3 groups independently selected from —O—, substituted or unsubstituted —NH—, and —S—, a poly(substituted or unsubstituted (C₁-C₂₀)hydrocarbyloxy), and a poly(substituted or unsubstituted (C₁-C₂₀)hydrocarbylamino).

Biocatalysts (e.g., whole cells or purified enzymes) hold great promise, due to their abilities to precisely perform chemical reactions. Biocatalysts have been developed for industrial scale production of a wide variety of commodity chemicals, pharmaceuticals, and industrial products as well as for biodegradation of pollutants from the environment. The efficiency and specificity with which biocatalysts can perform reactions can make them desirable compared to conventional reactants and catalysts used in synthetic chemistry. However, the high degree of specificity can make it necessary to discover or develop new enzymes for a desired reaction if one does not already exist, long term stability can be a challenge, and a solid support system can be needed to retain the biocatalyst within a reactor, all of which contribute costs which may exceed that of a conventional process. The use of whole cell biocatalysts, rather than purified enzyme, can significantly reduce the cost, but have reaction rates 1-2 orders of magnitude lower, due to the limited permeability of the cell envelope.

Layer-by-layer (LbL) self-assembly can have the potential to reduce cost and increase the functionality of whole cell biocatalysts. LbL coatings can protect the biocatalysts, provide long-term stability, and immobilize them within a bioreactor. While there are a variety of methods to create a LbL assembly, (electrostatic, hydrogen bonding, covalent, biological, hydrophobic, etc.) the use of oppositely charged polyelectrolytes via electrostatic interaction is desirable. The layer size, coating thickness, and porosity can be precisely controlled with the polyelectrolyte charge density, which is a function of pH, ionic strength, solvent quality, temperature, and molecular weight.

Various embodiments of the present disclosure relate to a biological assembly for performing biocatalysis. The assembly includes, at least, a biological component and a porous shell layer. The porous shall layer at least partially coats the biological component. For example, the porous shell can layer can coat about 50% surface area to about 100% surface area of the biological component, about 80% surface area to about 100% surface area, or less than, equal to, or greater than about 50% surface area, 55, 60, 65, 70, 75, 80, 85, 90, 95, or about 100% surface area.

The biological component of the assembly can be chosen from many suitable biological component. Examples of suitable biological components include a cell, an enzyme, a protein, an organelle, a liposome, a lipid membrane, or a mixture thereof. The assembly can include one of the components or a plural number. For example, the assembly can include one cell or a plural number of cells that are at least partially coated by the porous shell layer. In examples where the biological component is a cell, the cell can be a eukaryotic cell or a prokaryotic cell. An example of a suitable eukaryotic cell can include a mammalian cell or yeast. An example of a suitable prokaryotic cell can include a bacteria such as a gram-positive bacteria and a gram-negative bacteria.

The bacteria can he present as single bacterial cell or a colony. In examples, including more than one bacterial cell the bacteria may the same species of a mixture of different species. Examples of suitable gram-negative bacteria species include Acetic acid bacteria, Acidaminococcus, Acinetobacter baumannii, Agrobacterium tumefaciens, Akkermansia muciniphila, Anaerobiospirillum, Anaerolinea thermolimosa, Anaerolinea thermophila, Arcobacter, Arcobacter skirrowii, Armatimonas rosea, Azotobacter salinestris, Bacteroides, Bacteroides fragilis, Bacteroides ureolyticus, Bacteroidetes, Bartonella japonica, Bartonella koehlerae, Bartonella taylorii, Bdellovibrio, Brachyspira, Bradyrhizobium japonicum, Caldilinea aerophile, Cardiobacterium hominis, Chaperone-Usher fimbriae, Christensenella, Chthonomonas calidirosea, Coxiella burnetiid, Cyanobacteria, Cytophaga, Dehalogenimonas lykanthroporepellens, Desulfurobacterium atlanticum, Devosia pacifica, Devosia psychrophila, Devosia soli, Devosia subaequoris, Devosia submarina, Devosia yakushimensis, Dialister, Dicryoglomus thermophilum, Enterobacter, Enterobacter cloacae, Enterobacter cowanii, Enterobacteriaceae, Enterobacteriales, Escherichia, Escherichia coli, Escherichia fergusonii, Escherichia hermannii, Fimbriimonas ginsengisoli, Flavobacterium, Flavobacterium akiainvivens, Francisella novicida, Fusobacterium necrophorum, Fusobacterium nucleatum, Fusobacterium polymorphum, Haemophilus felis, Haemophilus haemolyticus, Haemophilus influenzae, Haemophilus pittmaniae, Helicobacter, Kingella kingae, Klebsiella pneumoniae, Kluyvera ascorbate, Kluyvera cryocrescens, Legionella, Legionella clemsonensis, Legionella pneumophila, Leptonema illini, Leptotrichia buccalis, Levilinea saccharolytica, Luteimonas aquatic, Luteinonas composti, Luteimonas lutimaris, Luteimonas marina, Luteimonas mephitis, Luteimonas vadose, Megamonas, Megasphaera, Meiothermus, Meiothermus timidus, Methylobacterium fujisawaense, Morax-Axenfeld diplobacilli, Moraxella, Moraxella bovis, Moraxella osloensis, Morganella morganii, Mycoplasma spumans, Neisseria cinereal, Neisseri gonorrhoeae, Neisseria meningitidis, Neisseria polysaccharea, Neisseria sicca, Nitrosomonas eutropha, Nitrosomonas halophila, Nonpathogenic organisms, OMPdb, Pectinatus, Pedobacter heparinus, Pelosinus, Propionispora, Proteobacteria, Proteus mirabilis, Proteus penneri, Pseudomonas, Pseudomonas aeruginosa, Pseudomonas luteola, Pseudoxanthomonas broeghernensis, Pseudoxanthomonas japonensis, Rickettsia rickettsia, Salinibacter ruber, Salmonella, Salmonella bongori, Salmonella enterica, Samsonia, Selenomonadales, Serratia marcescens, Shigella, Shimwellia, Solobacterium moorei, Sorangium cellulosum, Sphaerotilus natans, Sphingomonas gei, Spirochaeta, Spirochaetaceae, Sporomusa, Stenotrophomonas, Stenotrophomonas nitritireducens, Thermotoga neapolitana, Thorselliaceae, Trimeric autotransporter adhesion, Vampirococcus, Verminephrobacter, Vibrio adaptatus, Vibrio azasii, Vibrio campbellii, Vibrio cholerae, Victivallis vadensis, Vitreoscilla, Wolbachia, Yersiniaceae, Zymophilus, strains thereof, or combinations thereof.

In examples where the biological component includes an organelle, the organelle can be chosen from any suitable eukaryotic or prokaryotic organelle. Examples of organelles include a mitochondria, a nucleolus, a nucleus, a ribosome, a vesicle, an endoplasmic reticulum, a Golgi apparatus, a vacuole, a lysosome, a centrosome, or a combination thereof.

In examples where the biological component includes a protein or an enzyme, any suitable protein or enzyme can be included. Selection of the appropriate protein or enzyme can be driven by the desired functionality of the assembly (e.g., a desired reaction or process that is facilitated by the biological component). Examples of suitable proteins or enzymes can include atrazine chlorohydrolase (AtzA), lipase, nitrilase, amidase, acylase, adolase, transaminase, hydroxylase, and dioxygenase.

In some embodiments, the biological component may be chosen due to its catalytic ability. In these embodiments, the biological assembly functions to facilitate the biocatalysis of a desired reaction. The reaction can he one used, for example, to produce biological precursors, biopolymers, pharmaceuticals, industrial products. While the assembly can catalyze any desired reaction, an example of a suitable reaction includes the catalysis of 3,4-dihydroxyphenylacetic acid (DHPAC) to α-hydroxy δ-carboxymethyl cis-muconic semialdehyde. Another example of a suitable reaction includes the conversion of atrazine to hydroxyatrazine. In that reaction the biological component can be atrazine chlorohydrolase (AtzA)

According to various embodiments, an individual porous shell layer includes an inorganic network. The inorganic network can he an ionic network including a cationic component and an anionic component. The assembly can include any suitable plural number of porous shell layers. While the upper boundary of layers does not need to be limited, as an example, the assembly can include from 1 to 30 porous shell layers, 3-15, 4-8, or less than, equal to, or greater than 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 porous shell layers. With respect to each other, adjacent porous shell layers are held in contact through a hydrogen bond, an electrostatic interaction, an ionic bond, a covalent bond, adhesion, a physical interlocking connection, and a combination thereof.

Different porous layers can include the same cationic and anionic components with respect to each other. Alternatively, different porous layers can include different cationic and anionic components with respect to each other. Selection of specific cationic components and anionic components can be driven by factors such as the reactivity of each component with respect to one another and the physical properties (e.g., porosity, strength, thickness) of the resulting inorganic network.

Examples of suitable anionic components include an acidified silane. Suitable acidified silanes can include tetramethylorthosilicate (TMOS), tetraethylorthosilicate (TEOS), tetrakis(2-hydroxyethyl)orthosilicate (THEOS), rnethyldiethoxysilane (MDES), 3-(glycidoxypropyl)triethoxysilane (GPMS), 3-(trimethyoxysilyl)propylacrylate (TMSPA), N-(3-triethoxysilylpropyl)pyrrole (TESPP), vinyltriethyoxysilane (VTES), methacryloxypropyltriethoxysilane (TESPM), diglycerylsilane (DOS), methyltriethoxysitane (MTMOS), trimethylmethoxysitane (TMMS), ethyltriethoxysilane (TEES), n-propyltriethoxysilane (TEPS), n-butyltriethyoxysilane (TEBS), 3-aminopropyltriethoxysilane (APTS), 2-(2,4-dinitrophenylamino)propyltriethoxysilane, mercaptopropyltriethoxysilane (TEPMS), 2-(3-aminoethylamino)propyltriethoxysilane, isocyanatopropyltriethoxysilane, hydroxyl-terminated polydimethyisiloxane, triethoxysityl-terminated polydimethylsiloxane, methyltriethoxysilane (MTES), triethoxysilyl-terminated poly(oxypropylene), or a mixture thereof. The cationic component can be any cationic component that is capable of forming the inorganic network with the anionic component.

Examples of suitable cationic components include any cationic polymer or molecule that differs in at least one of charge or hydrophobicity from the anionic component. Exampels of suitable polymers include poly(allylamine hydrochloride) (PAH), poly(4-vinylpyridine) (P4VP), poly acrylic acid (PAA), or a combination thereof. Further examples of suitable cationic components include a quaternary ammonium salt. Suitable examples of quaternary ammonium salts include quaternary ammonium salts having the structure according to Formula I:

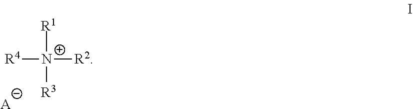

In Formula I, R¹, R², R³, and R⁴ are independently selected from the group consisting of —H, substituted or unsubstituted (C₁-C₂₀)alkyl, (C₂-C₂₀) alkenyl, (C₁-C₂₀)acyl, (C₄-C₂₀)cycloalkyl, (C₄-C₂₀)aryl, and combinations thereof. Furthermore, in Formula I, A is selected from the group consisting of F⁻, Cl⁻, Br⁻, and I⁻. In further examples, the quaternary ammonium salt has the structure according to Formula II:

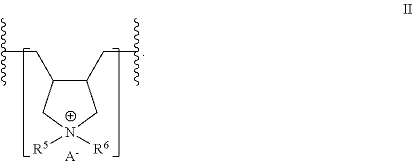

In Formula II, R⁵ and R⁶ are independently selected from the group consisting of —H, substituted or unsubstituted (C₁-C₂₀)alkyl, (C₂-C₂₀)alkenyl, (C₁-C₂₀)acyl, (C₄-C₂₀)cycloalkyl, (C₄-C₂₀)aryl, and combinations thereof. Furthermore in Formula II, A is selected from the group consisting of F⁻, Cl⁻, Br⁻, and I⁻. Examples of specific quaternary ammonium salts include those chosen from acetylcarnitine, acetylcholine, aclidinium bromide, acriflavine, agelasine, aliquat 336, ambenonium chloride, ambutonium bromide, nnine-6plus atracurium besilate, behentrimonium chloride, benzalkonium chloride, benzethonium chloride, benzilone, benzododecinium bromide, benzoxonium chloride, benzyltrimethylammonium fluoride, benzyltrimethylammonium hydroxide, bephenium hydroxynaphthoate, berberine, betaine, bethanechol, bevonium, bibenzonium bromide, bis-choline tetrathiomolybclate, bretylium, bufothionine, butyrylcholine, californidine, candicine, candocuronium iodide, carbachol, carbethopendecinium bromide, carnitine, cefluprenam, cetalkonium chloride, cetrimonium, cetrimonium bromide, cetrimonium chloride, chelerythrine,chlorisondamine, chlormequat, chotamine chloride hydrochloride, choline, choline chloride, eimetropium bromide, cisatracurium hesitate, citicoline, clidinium bromide, clofilium, cocamidopropyl betaine, cocamidopropyl hydroxysultaine, complanine, coptisine, cyanine, cyclobis(paraquat-p-phenylene), dacuronium bromide, decamethonium, 3-dehydrocarnitine, demecarium bromide, denatonium, dequalinium, didecyldimethylammonium chloride, dihydrochandonium, dimethyldioctadecylammonium chloride, dimethylphenylpiperazinium, dimethyltubocurarinium chloride, diphemanil metilsulfate, diphthamide, diquat, dithiazanine iodide, domiphen bromide, doxacurium chloride, echothiophate, edelfosine, edrophonium, emepronium bromide, ethidium bromide, ethyl green, euflavine, fenpiverinium, fentonium, fluorocholine, gallamine triethiodide, gantacurium chloride, glycine betaine aldehyde, glycopyrronium bromide, guar hydroxypropyltrimonium chloride, hemicholinium-3, hexafluronium bromide, hexamethonium, hexocyclium, hydroxyethylpromethazine, hyoscine butylbromide, ipratropium bromide, isometamidium chloride, isopropamide, jatrorrhizine, laudexium metilsulfate, lucigenin, meldonium, mepenzolate, methacholine, methantheline, methiodide, methylatropine, methylhomatropine, methylnaltrexone, methylscopolamine bromide, metocurine, miltelosine, morphine methylbromide, muscarine, neurine, obidoxime, octatropine methylbromide, octenidine dihydrochloride, otilonium bromide, oxapium iodide, oxitropium bromide, oxyphenonium bromide, pahutoxin, palmatine, pancuronium bromide, pararosaniline, pentamine, penthienate, pentolinium, perifosine, phellodendrine, phosphocholine, pinaverium, pipecuronium bromide, pipenzolate bromide, poldine, polydadmac, polyquaternium, polyquaternium-7, pralidoxime, prifinium bromide, propantheline bromide, prospidium chloride, pyrvinium, quaternium-15, quinapyramine, rapacuronium bromide, rhodamine b, rimazolium, rocuronium bromide, safranin, sanguinarine, selectfluor, silane quats, sinapine, stearalkonium chloride, stercuronium iodide, succinylmonocholine, suxamethonium chloride, suxethonium chloride, tetra-n-butylammonium bromide, tetra-n-butylammonium fluoride, tetrabutylammonium, tetrabutylammonium hexafluorophosphate, tetrabutylammonium hydroxide, tetrabutylammonium tribromide, tetraethylammonium, tetraethylammonium bromide, tetraethylammonium chloride, tetraethylammonium iodide, tetramethylammonium, tetramethylammonium chloride, tetramethylammonium hydroxide, tetramethylammonium pentafluoroxenate, tetraoctylammonium bromide, tetrapropylammonium perruthenate, thiazinamium metilsulfate, thioflavin, thonzonium bromide, tibezonium iodide, tiemonium iodide, timepidium bromide, toxiferine, trazium, tridihexethyl, triethylcholine, trigonelline, trimethylglycine, trolamine salicylate, trospium chloride, tubocurarine chloride, umeclidinium bromide, vecuronium bromide, and mixtures thereof.

An individual porous shell layer can have any desired level of porosity. For example, a porosity of the individual porous shell layers can range from about 5% by volume to about 80% by volume, about 10% to about 30%, or less than, equal to, or greater than about 5%, 10, 15, 20, 25, 30, 35, 40, 45, 50, 55, 60, 65, 70, 75, or about 80% by volume. An individual pore can be tuned to have a diameter in major dimension across a width of the of any suitable value. As a general consideration, the diameter of an individual pore can be sized to be large enough to allow a substance to pass through the porous shell layer to come into contact with the biological component, yet small enough to not allow the biological component to fit through and exit the assembly.

The individual porous shell layers can have a uniform or variable thickness. On average, the thickness of an individual porous shell layer can be in the range of from about 0.5 nm to about 50 nm, about 3 am to about 10 nm, or less than, equal to, or greater than about, 0.5, 1, 5, 10, 15, :20, 25, 30, 35, 40, 45, or 50 nm.

The individual porous shell layers can serve many functions in the assembly. An example of one such function is that the layers can help to increase the permeability of a cell membrane to allow for ingress of substances or reactants into the cell. Another function is that the individual layers serve as protection for the biological component from external antagonists and during storage generally. While adding more layers may enhance protection there is a chance that permeability can be decreased with additional layers. Conversely fewer layers can enhance permeability, yet decrease protection. Without wishing to be bound to any particular number, the inventors have found that about 3-6 layers provides a suitable balance between these factors. in some examples, it is possible to build an excess of porous layers for the purpose of successfully storing the assembly. The layers, or portions thereof, can be removed from the assembly to increase permissibility in the assembly prior to use.

The assembly can be modified further to enhance any one of permeability and protection. For example, permeability can be enhanced by contacting at least a portion of any one of the porous shell layers with a detergent or surfactant. Examples of suitable detergents or surfactants include deoxycholic acid, sodium lauroyl sarcosinate, polysorbate 85 (Tween 85), and polyethylene glycol p-(1,1,3,3-tetramethylbutyl)-phenyl ether, and mixtures thereof.

The biological component can be further protected in the assembly by modifying the biological component. For example, if the biological component includes an enzyme, a protein, or an organelle, they can be tethered or grafted to a substrate. An example of a suitable substrate includes a granulated activated carbon, glass, plastic, ceramic, metal, or a combination thereof. In some examples the substrate is, or is a component of, a bioreactor the reaction chamber), a well (e.g., or a well-plate), or a paper strip (e.g., a wicking strip). Additionally, or alternatively, the enzyme, protein, or organelle, can be at least partially surrounded by a lipid bilayer. The lipid bilayer, in turn, can be in contact with at least one of the porous shell layers.

The ability to tune the permeability and protective characteristics of the biological assembly can allow the biological structure substantially retain the ability to perform biocatalysts under wide variety of circumstances. For example, hiocatalysis can be maintained, at least to a sufficient level, when the assembly is subjected to osmotic shock, lysozyme treatment, desiccation, freezing, thawing, heating, exposure to protozoa, or combinations thereof. Conditions correlating to osmotic shock can be met, for example, by exposing the assembly to a solution comprising ethylenediaminetetraacetic acid (EDTA) and 40% (w/v) sucrose in polybutylene. Conditions correlating to lysozyme treatment can include, for example, incubating the assembly in a mixture comprising ethylenediaminetetraacetic acid (EDTA) and lysozyme at room temperature. Conditions correlating to desiccation can include, for example, drying the assembly at a vacuum pressure of about 0 Pa to about 5 Pa, about 0.5 Pa to 4 Pa, about 1 Pa. to about 3.5 Pa, or less than, equal to, or greater than about 0 Pa, 0.5, 1.5, 2, 2.5, 3, 3.5, 4, 4.5, or about 5 Pa. Conditions correlating to freezing can include, for example, lowering a temperature of the assembly to a temperature of about 0° C. to about −190° C., −5° C. to about −90° C., −20° C. to about −50° C., or less than, equal to, or greater than about 0° C., −5, −10, −15, −20, −25, −30, −35, −40, −45, −50, −55, −60, −65, −70, −75, −80, −85, −90, −95, −100, −105, −110, −115, −120, −125, −130, −135, −140, −145, −150, −155, −160, −165, −170, −175, −180, −185, or −190° C. Conditions correlating to heating can include, for example, heating the assembly from above room temperature to about 100° C., to no greater than about 5° C. lower than a denaturization temperature of the biological assembly. For example, heating can include raising the temperature to from just above room temperature to those where extremophite thrive such as about 30° C. to about 500° C., about 40° C. to about 100° C., or less than, equal to, or greater than about 30° C., 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 3′70, 380, 390, 400, 410, 420, 430 440, 450, 460, 470, 480, 490, or about 500° C.

Conditions correlating to exposing the biological assembly to protozoa can include, for example, incubating the assembly with a protozoa chosen from Amoeba, Paramecium, Chilomonas, Stentor, Euglena, Volvox, or a combination thereof.

The assembly can be made using many suitable methods. In a suitable method of the disclosure refered to as a layer-by-layer (LbL) processes, the biological component can be contacted with an acidified anionic component simultaneously or shortly thereafter the cationic component can be added to form the inorganic network of the individual porous layer. In some embodiments, it may be desirable to add the anionic component first, to increase the permeability of the resulting porous layer. In other examples, however, it may be desirable to add the cationic component before adding the anionic component. The process of adding the respective anionic and cationic components can be repeated to form subsequent porous shell layers.

### EXAMPLES

Various embodiments of the present disclosure can be better understood by reference to the following Examples which are offered by way of illustration. The present disclosure is not limited to the Examples given herein.

### Example 1

### Materials

Tetramethoxysilane (TMOS, 98% purity), poly(diallyldimethylammonium chloride) (20 wt % in H₂O, average M_{w} 200,000-350,000), 3,4-dihydroxyphenylacetate (DHPAC), and all other chemicals were purchased from Sigma-Aldrich (Sigma-Aldrich Corp., St. Louis, Mo., USA) and used without further purification. Ultrapure water (UPW) was prepared by filtering distilled water using a Milli-Q water purification system (Millipore, Billerica, Mass., USA) to a final electrical resistance of >18.2 MΩ/cm.

### Example 2

### Bacterial Strains and Growth Conditions

Homoprotocatechuate 2,3-dioxygenase isolated from Brevibacterium fuscum (2,3-HPCD) was expressed in E. coli DH5α grown overnight on Luria Broth (LB) with 100 μg/mL ampicillin. In some cases, GFP-expressing E. coli DH5α were grown on LB with 100 μg/mL kanamycin. E. coli cells were harvested by centrifugation at 5,000×g for 10 mins before resuspension in 50 mM sodium phosphate buffer at pH 5.8 (PB).

### Example 3

### Layer-by-Layer (LbL) Deposition

A cell suspension at either 2 or 20 μg/mL was aliquoted (50 into the wells of a 96-well plates. For all wells except those for control, 200 μL of 0.5% PDADMAC in 150 mM NaCl was added and incubated for 5 mins. The plates were centrifuged at 3,000 rpm for 30 mins to deposit the cells into films on the bottom of the plates. The supernatant was removed and. the wells were washed 2× with PB before adding 200 μL of 100 mM silicic acid solution for 10 mins, followed by washing 2× with PB. The silicic acid solution was prepared by the hydrolysis of 1 M TMOS in 5 mM HCl for 20 mins before 1:9 (v/v) dilution to 100 mM. TMOS in PB at pH 5.8. The process of PDADMAC/SiO₂ deposition was repeated up to 10 times for the desired number of layers.

### Example 4

### Microstructural Analysis

Samples for examining the deposition of material onto the bacteria were made on silicon chip supports. The bacteria suspension was incubated on the silicon for 30 mins before washing off unattached cells with PB. The LbL process was performed by dipping the silicon chips into reservoirs containing either hydrolyzed TMOS, PDADMAC, or PB, as described herein, until the desired number of layers were achieved. The microstructure was examined with a Hitachi SU-8230 field emission gun scanning electron microscope (SEM, Hitachi, Ltd., Tokyo, Japan). The samples were fixed for up to 20 hrs with 2% para-formaldehyde (EM grade), 2% glutaraldehyde (EM grade), and 4% sucrose in 0.14 M sodium cacodylate buffer at pH 7.4. After fixation, the samples were washed 3× with buffer before adding the postfix solution of 1% OsO₄ and 1.5% potassium ferrocyanide in 0.15 M sodium cacodylate. The samples were incubated in the postfix solution for 90 mins while occluded from light. After postfixation, the samples were washed 3× and gradually dried in increasing ethanol concentrations of 50%, 75%, 90%, and 100% (twice) for 10 minutes per wash. The samples were then critical-point dried using a Tousimis Model 780A Critical-Point Dryer before being coated with 5 nm of Ir using a Leica EM ACE600 sputter coater. Energy dispersive x-ray spectroscopy (EDS) was performed using a Noran System 7 detector (Thermo Scientific, Waltham, Mass., USA).

### Example 5

### Permeability Measurement

The permeabilities of 1-N-phenylnaphthylamine (NPN) and propidium iodide (PI) were measured. To measure the permeability, 200 μL aliquot of 5 μM NPN and PI was added to each well. The fluorescence intensity of NPN (λₑₓ=348 nm, λₑₘ=408 nm) and PI (λₑₓ=535 nm and λₑₘ=617 nm) were measured for up to one hour with a SpectraMAX EM (Molecular Devices, Sunnyvale, Calif., USA). The relative changes in fluorescence followed first order reaction kinetics as given by:

$$\frac{d F}{d t} = {k}_{p} ( {F}_{max} - F ) \qquad ( 1 )$$

Transforming the fluorescence intensity:

$$f ( t ) = \frac{F ( t ) - F ( 0 )}{{F}_{max}} \qquad ( 2 )$$

And finally, the double normalized fluorescence intensity f(t) is:

f(t)=1−e^{-k}^{ₚ}^{t} (3)

The experimental data was fit to Equation 3 with a least squares regression to determine the respective first order permeability constants (kₚ).

### Example 6

### Biocatalytic Activity Measurement

The activity of DHPAC conversion to α-hydroxy δ-carboxymethyl cis-muconic semialdehyde was measured by the absorption of the product at 380 nm (A₃₈₀). A 200 μL aliquot of 500 μM DHPAC in 50 mM PB at pH 7.5 was added to each well and A₃₈₀ was measured for up to one hour. The data was then fit to a first order reaction model of the form:

Cₚ(t)=C_{s,0}(1−e^{-k}^{ᵣ}^{t}) (4)

Cₚ is the product concentration, C_{s,0} is the initial substrate concentration, and kᵣ is the first order reaction rate constant.

### Example 7

### Detergent/Surfactant Treatment

The coated and uncoated samples were treated with a variety of surfactants or detergents, including: deoxycholic acid (sodium salt), sodium lauroyl sarcosinate (Sarkosyl), polysorbate 85 (Tween 85), and Triton X-100. A 200 μL aliquot of the desired detergent at 1% concentration (w/v) in water was added to each well and incubated for 20 mins at room temperature before washing 5× with PB.

TABLE 1
Detergent and micelle properties

| | | | | Est. | |
|---|---|---|---|---|---|
| | | | | Micelle | |
| | | MW | Aggregation | MW | CMC |
| Detergent | Type | (Da) | Number | (kDa) | (M) |
| Deoxycholic | Bile Salt | 393 | 22 | 9 | 3 × 10⁻³ |
| Acid | | | | | |
| Sodium Lauroyl | Anionic | 293 | 2 | <1 | |
| Sarcosinate | | | | | |
| Triton X-100 | Nonionic | 1625 | 140 | 228 | 3 × 10⁻⁴ |
| Tween 85 | Nonionic | 1839 | 60 | 110 | 1.2 × 10⁻⁵ |
| | | | (Tween 80) | | |

### Example 8

### Osmotic Shock

Samples were exposed to osmotic shock. PB was added to LbL samples with up to 10 layers before a 50% dilution with a solution containing 1 mM ethylenediaminetetraacetic acid (EDTA) and 40% (w/v) sucrose in PB. The samples were incubated for 10 mins to allow for sucrose uptake. The supernatant was then removed and replaced with ice cold DI water for 10 mins. Finally, the DI water was removed and the samples were washed before activity measurement.

### Example 9

### Lysozyme Treatment

LbL samples were incubated in 1 mM EDTA with 10 μg/mL lysozyme for 1 hr at room temperature, then washed with PB before activity measurement.

### Example 10

### Desiccation

LbL samples were washed, the supernatant was removed, and the samples were allowed to briefly air dry. The samples were then vacuum dried for 24 hrs at a vacuum pressure of 1 Pa. After drying, the samples were rehydrated with PB before activity measurement.

### Example 11

### Freeze/Thaw

LbL samples were frozen at −20° C. for 1 hr and washed with PB before activity measurement.

### Example 12

### Heat Treatment

Two distinct heat treatment experiments were conducted. High temperature incubation for 30 mins at 54° C. prior to activity measurement at room temperature. In the second experiment, the samples were assayed at elevated temperatures.

### Example 13

### Protozoa Exposure

LbL samples with up to 10 layers were incubated with mixed protozoa containing Amoeba, Paramecium, Chilomonas, Stentor, Euglena, and Volvox (Carolina Biological, Burlington, N.C.) overnight before washing and activity measurement.

### Example 14

### Protein Retention Over Tim

GFP-expressing cells were deposited into a film with up to 10 layers. The GFP fluorescence of the film was measured with λₑₓ=480 nm, λₑₘ=510 nm, and a 495 nm cutoff filter. The samples were stored at room temperature in PB and washed before measurement.

### Example 15

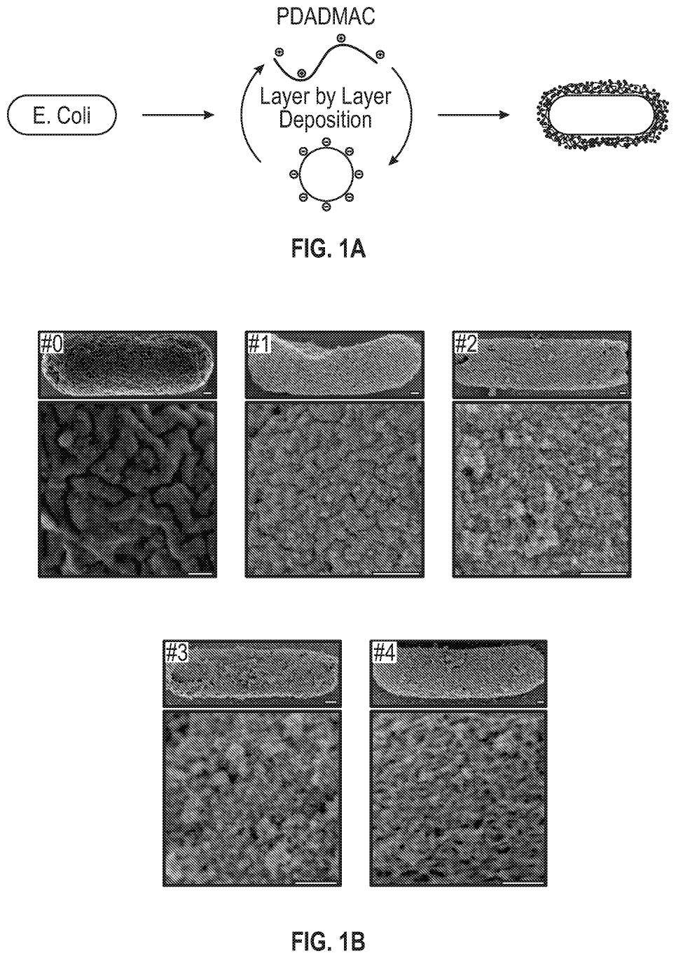

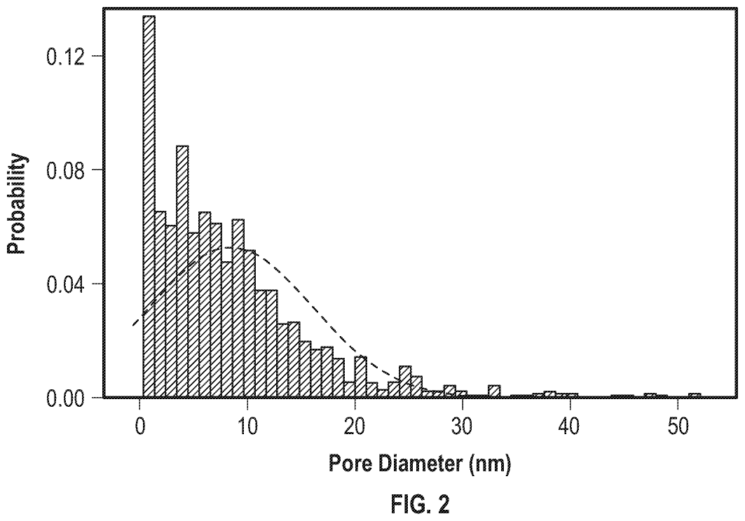

The microbial exoskeleton for E. coli was constructed as shown in FIG. 1A, using an LbL coating process. FIG. 1A shows a diagram of the layer by layer coating process involved in the creation of the microbial exoskeleton by alternating deposition of poly(diallyldimethylammonium chloride) (PDADMAC) and hydrolyzed tetramethyl orthosilicate (TMOS) a silica precursor. By alternating between exposure to the polycation PDADMAC and SiO₂, which is negatively charged above the isoelectric point of pH 1.8, the thickness of the exoskeleton could be precisely controlled. The bacteria were imaged with SEM after deposition of each layer, generating a sequence of images, which showed a gradual disappearance of the outer membrane structure after coating with a first couple of layers. This is shown in FIG. 1B, which are SEM images of E. coli cells with increasing number of SiO₂/PDADMAC layers from 0 to 4 (left to right). In FIG. 1B, bottom panels show a magnified view. Scale bars are 100 nm. After 4 or more layers of coating, a relatively uniform silica coating was observed, with pores on the order of 10 nm, this is shown in FIG. 2, which is a graph showing apparent pore diameter measured from SEM images, n>1,300. In that figure, the dashed red line indicates a probability density function fit to the mean and standard deviation.

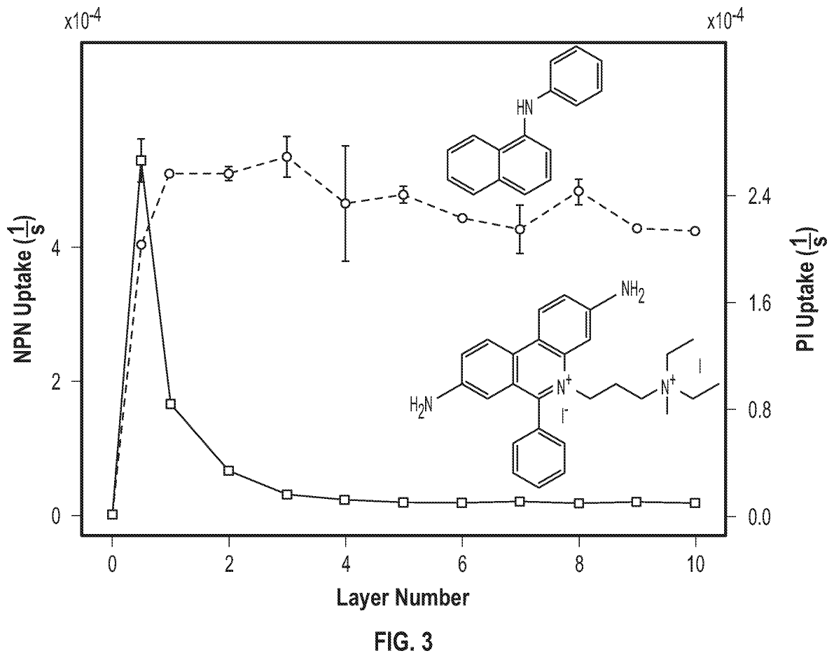

The effect of LbL deposition on the permeability of the cells was determined using the fluorescent probes NPN and PI. FIG. 3 is a graph showing the effect of number of layers deposited on cell envelope permeability to 1-N-phenylnaphthylamine (NPN), and propidium iodide (PI), n=4. Layer #0.5 corresponds to PDADMAC coating only. Chemical structures of NPN and PI shown above each dataset. As shown, a significant increase in the permeability of the membrane to NPN was observed after coating the cell with PDADMAC (layer #0.5). The permeability remained relatively constant regardless of the number of layers deposited. NPN fluoresces in a hydrophobic microenvironment, but is not cell permeable. These results suggested that PDADMAC disrupted the lipopolysaccharide (LPS) layer, allowing NPN to access the outer membrane (OM), and are consistent with the reports of polycation-mediated disruption of the OM. Since PI must pass through the LPS, OM, and the cytoplasmic membrane (CM) in order to bind with the nucleic acids, it was used to help distinguish OM and CM disruption when compared with NPN. Permeability of the membrane to PI also increased rapidly with the addition of PDADMAC, but tapered as a function of the number of layers deposited. PDADMAC seemed to disrupt the CM only within the first few layers. This may suggest structural instability between the OM, CM, and lipoprotein anchor points, which connect the OM and CM to the cell wall. Since the PI permeability returns to nominal after 3-4 layers, it may indicate that the PDADMAC/SiO₂ layers re-stabilize the cell envelope and close any holes within the membrane after sufficient exoskeleton coverage. Another factor could be the increasing mass transport length scale due to increasing exoskeleton shell thickness, though this effect was expected to he minimal on the length scale of ˜3-30 nm.

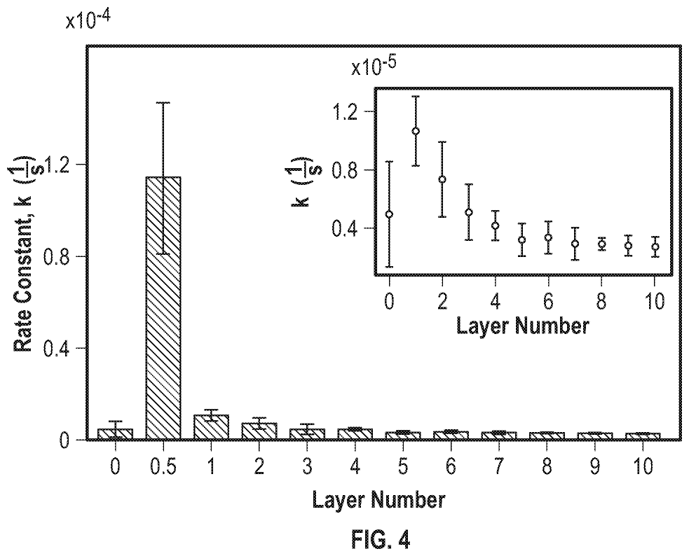

The effect of the microbial exoskeleton on biocatalysis was investigated using homoprotocatechuate 2,3-dioxygenase (HPCD) conversion of 3,4-dihydroxyphenylacetate (DHPAC). An increase of more than an order of magnitude in the reaction rate constant was observed when the cells were coated with a PDADMAC layer. This is shown in FIG. 4, which is a graph showing the effect of the number of layers on biocatalytic activity with homoprotocatechuate 2,3-dioxygenase (HPCD), n=16. In FIG. 3 the inset has Layer #0.5 (PDADMAC only) data removed to better highlight the small changes observed in rate constant with increased number of layers. When comparing the rate constants of full layers (PDADMAC/SiO₂) only, an increase was still observed in the first few layers, with a gradual decline in a layer-dependent manner. Since the ring opened product of DHPAC biocatalysis was measured in the supernatant, rather than within the film, the reported rate constant included not only the rate of the reaction but also the transmembrane transport of the substrate and product. Both the substrate and product are hydrophilic, and in lieu of porin-mediated transport, the rate limiting steps are therefore expected to be the transport through the lipid bilayers including the OM and CM. For hydrophobic molecules (such as NPN, however), the LPS layer is expected to provide a larger barrier to transport. The reaction rate constant of the coated cells was more than an order of magnitude faster than free cells in suspension, but still nearly an order of magnitude slower than that of the free enzyme. This is shown in FIG. 4, which is a graph showing a comparison of the reaction rate of the free enzyme from lysed cells, free cells in suspension, and cells with microbial exoskeleton (SiO₂/PDADMAC) n=16. This suggested that a significant harrier to mass transport remained, despite the increased permeability of the OM.

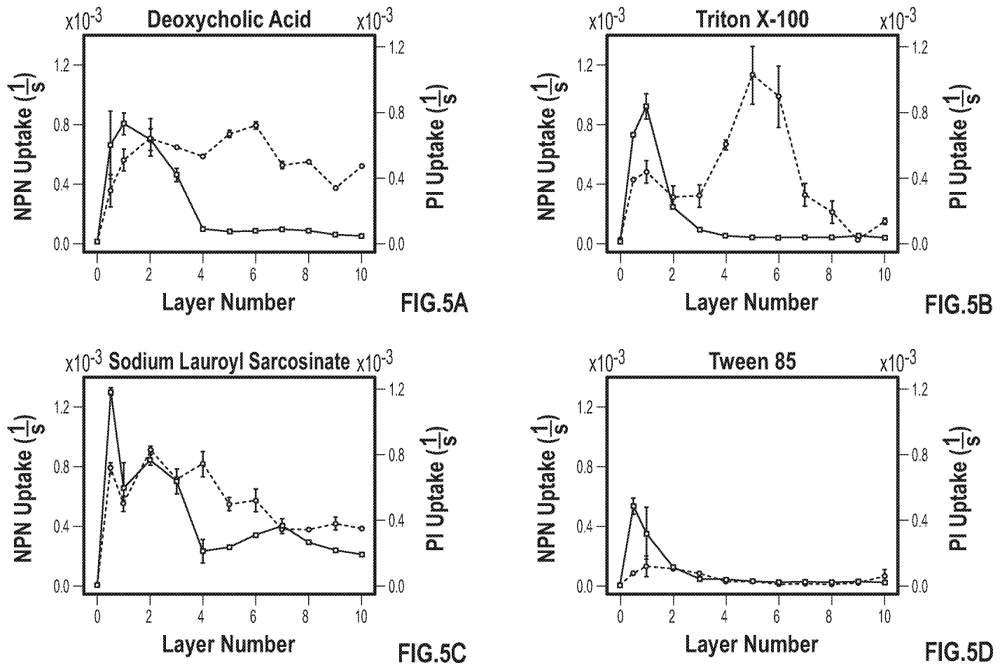

The inventors hypothesized that surfactant treatment could be used to specifically target the CM to determine if indeed that was the rate limiting factor for biocatalysis. Several commonly used surfactants or detergents were investigated for further enhancement of biocatalysis. The three types of detergents used were bile salts (Deoxycholic acid, DCA), anionic (Sodium lauroyl sarcosinate, Sarkosyl), and nonpolar (Triton X-100 and Tween 85, Table 1). The inventors suspected that targeting the CM should lead to a significant increase in the rate of biocatalysis, since the microbial exoskeleton already permeabilized the LPS/OM. Sarkosyl was chosen because it has been shown to specifically target the CM, and our results confirmed a large increase in the biocatalytic activity after Sarkosyl treatment. The permeability measurements showed little change in NPN uptake, while PI uptake increased dramatically, respective to untreated samples, which seemed to support the hypothesis that CM permeability was the main resistance to hiocatalysis in this study. While all the detergents tested led to increases in the rate of biocatalysis, DCA and Sarkosyl seemed to have much stronger effects than Triton X-100 and Tween 85. If indeed the PI uptake was a proxy for the CM permeability, then it remained unclear as to why the initial permeability with a monolayer of PDADMAC/SiO₂ showed significant increases which were not equally reflected in the rates of biocatalysis. Overall, the magnitude in the hiocatalysis rate changes seemed to correlate well with the detergent micelle sizes, in descending order: Sarkosyl>DCA>Tween 85>Triton X-100 (Table 1). Based on the observations of full microbial exoskeleton coverage after ˜4 layers and the sharp decline in PI uptake between layers 1-4, these results suggest that the larger detergent micelles, particularly Tween 85 and Triton X-100, were excluded from the coated cells. When compared with the size distribution of the exoskeleton pores, Triton X-100 micelles were very similar in size, with hydrodynamic radii ca 4 nm, while DCA micelles measured were much smaller, ca 1.3 nm. In summary, while these results showed that CM disruption was relevant in enhancing the reaction rates. FIGS. 5A-5D, are graphs showing the effect of various detergent/surfactant treatment (1% w/v for 20 minutes) on biocatalytic activity (n=6). While some increases were observed with all the surfactants tested, the enhancement observed with Tween 85 and Triton X-100 were modest in comparison. While these results showed that CM disruption was critical in restoring the reaction rate in samples containing SiO₂ layers to what was achieved with PDADMAC only (0.5 layers), in no case was a significant improvement on the reaction rate constant with PDADMAC only.

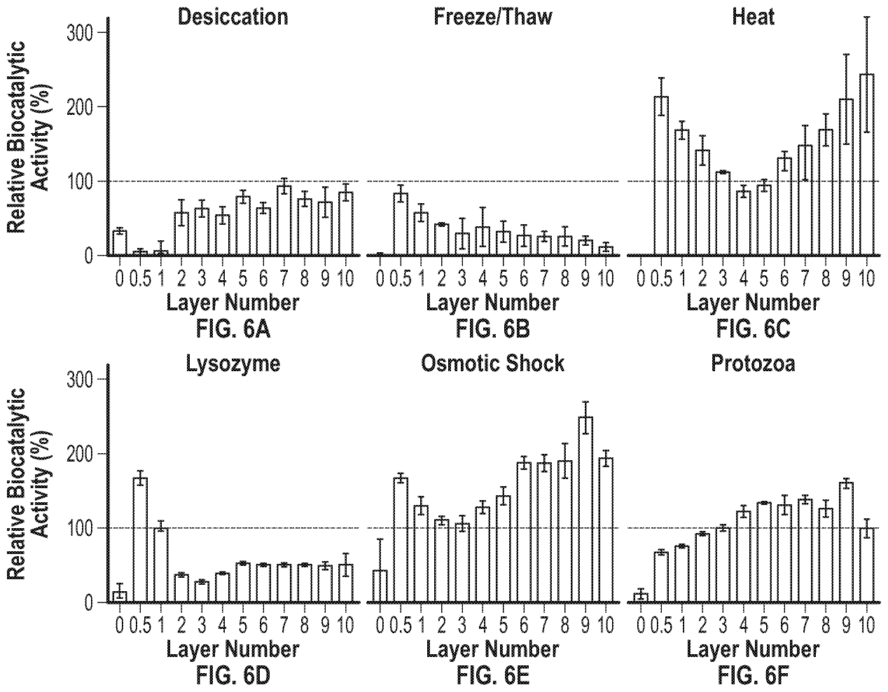

The ability of the microbial exoskeleton to protect the biocatalyst against a variety of stresses including exposure to high temperature, enzymatic attack, osmotic shock, freeze/thaw, desiccation, and predation, was examined. While any coating provided Unproved resistance over untreated cells (0 layers), freeze/thaw, high temperature treatment at 54° C., and lysozyme showed little difference when compared with their untreated counterparts. This is shown in FIGS. 6A-6F, which are a series of graphs showing protection offered by the exoskeleton against a variety of stresses. Exposure to; A) vacuum and desiccation; overnight at 10 Pa vacuum pressure (n=8), B) freeze/thaw with storage for 1 hr at −20° C. (n=4), C) high temperatures; 30 minutes at 54° C. (n=4), D) lysozyme at 10 mg/mL for 1 hr (n=4), E) osmotic shock with 40% sucrose+1 mM EDTA (1.2 Osm/L) for 20 mins (n=4), F) protozoa for 24 hrs (n=8). Given the minimal difference between lysozyme, and untreated cells, it was determined that the cell envelope, and therefore the cytoplasm remained intact. Interestingly, the osmotic shock treatment enhanced the reaction rate, which may be attributed to disruption of the CM. Desiccated samples retained nearly all of their initial activity, increasing with layer number. Finally, samples exposed to a variety of predatory protozoa maintained, and in some cases increased, their initial activity with a minimum of five layers, below which reduced activity was observed. This can be explained by the physical barrier that the microbial exoskeleton presents to the protozoa.

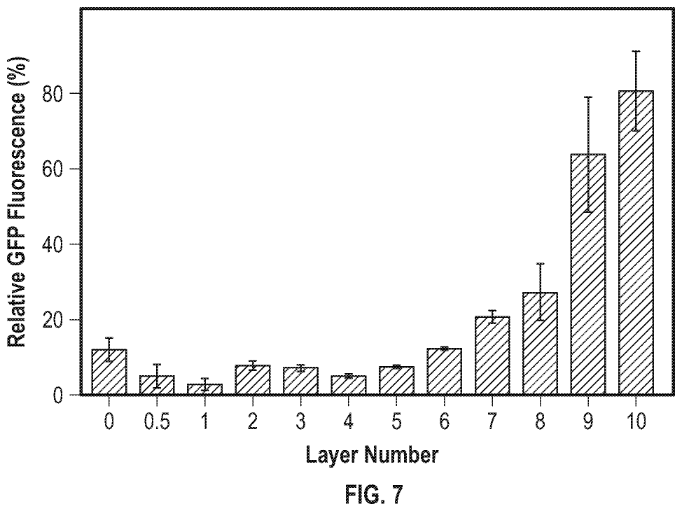

The ability of the microbial exoskeleton to retain protein after washing was also investigated. After five days of washing and incubation of the samples in PB a strong dependence on layer number was observed. This is shown in FIG. 7, which is a graph showing protein retention in phosphate buffer (pH 5.8) after 5 days with daily buffer washes to remove leaked protein (n=16). The results shown suggest that by permeabilizing the OM, and to some extent CM, was able to leak out of the treated cells. However, with a sufficient number of layers (>8), >80% of initial GFP fluorescence was observed, indicating that the coating had the ability to retain the protein. This may have been due to the physical barrier presented by the SiO₂ structure and tortuous nature of the path required for a protein to escape. Given the apparent size of the pores some protein may be able to escape over time, albeit very slowly, which correlated well with the observations between 1 and 5 days.

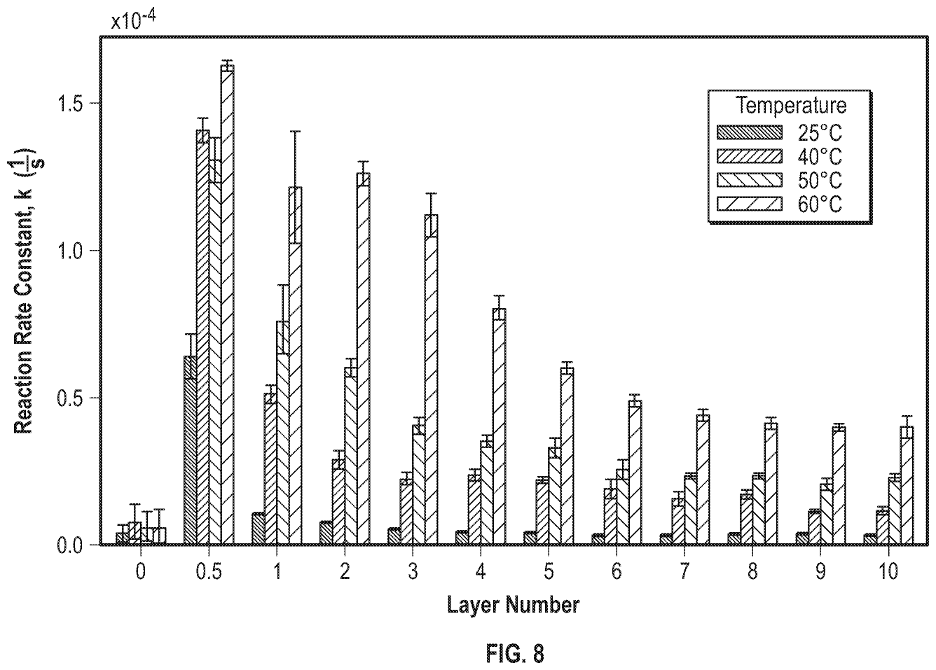

The use of the microbial exoskeleton to facilitate biocatalysis at elevated temperatures was also investigated. In nearly all cases, a temperature-dependent increase in the reaction rate constant was observed, which is consistent with previous work and theoretical predictions. This is shown in FIG. 8, which is a graph showing biocatalysis at elevated temperatures (n=4), In the case of untreated cells (0 layers), virtually no increase was observed. This may be due to untreated cells washing off the well plate after repeated assays. These results show the potential of using the microbial exoskeleton for biocatalysis at elevated temperatures, which can dramatically increase the throughput of a biocatalyst system.

The terms and expressions that have been employed are used as terms of description and not of limitation, and there is no intention in the use of such terms and expressions of excluding any equivalents of the features shown and described or portions thereof, but it is recognized that various modifications are possible within the scope of the embodiments of the present disclosure. Thus, it should be understood that although the present disclosure has been specifically disclosed by specific embodiments and optional features, modification and variation of the concepts herein disclosed may be resorted to by those of ordinary skill in the art, and that such modifications and variations are considered to be within the scope of embodiments of the present disclosure.

### Additional Embodiments

The following exemplary embodiments are provided, the numbering of which is not to be construed as designating levels of importance:

Embodiment 1 provides a biological assembly for performing biocatalysis, the assembly comprising:

a biological component; and

a porous shell layer at least partially coating the biological component, the porous shell layer comprising an inorganic network comprising a cationic component and an anionic component.

Embodiment 2 provides the biological assembly of Embodiment 1, wherein the biological assembly comprises at least one of a cell, an enzyme, a protein, and an organelle.

Embodiment 3 provides the biological assembly of Embodiment 2, wherein the cell is chosen from a eukaryotic cell, a prokaryotic cell, and a mixture thereof.

Embodiment 4 provides the biological assembly of Embodiment 3, wherein the eukaryotic cell is chosen from a mammalian cell, yeast, or a combination thereof.

Embodiment 5 provides the biological assembly of Embodiment 3, wherein the prokaryotic cell is a bacteria.

Embodiment 6 provides the biological assembly of Embodiment 5, wherein the bacteria is chosen from a gram-positive bacteria, a gram-negative bacteria, and a combination thereof.

Embodiment 7 provides the biological assembly of Embodiment 6, wherein the gram-negative bacteria is chosen from Acetic acid bacteria, Acidaminococcus, Acinetobacter baumannii, Agrobacterium tumefaciens, Akkermansia muciniphila, Anaerobiospirillum, Anaerolinea thermolimosa, Anaerotinea thermophila, Arcobacter, Arcobacter skirrowii, Armatimonas rosea, Azotobacter salinestris, Bacteroides, Bacteroides fragilis, Bacteroides ureolyticus, Bacteroidetes, Bartonella japonica, Bartonella koehlerae, Bartonella taylorii, Bdellovibrio, Brachyspira, Bradyrhizobium japonicum, Caldilinea aerophile, Cardiobacterium hominis, Chaperone-Usher fimbriae, Christensenella, Chthonomonas calidirosea, Coxiella burnetiid, Cyanobacteria, Cytophaga, Dehalogenimonas lykanthroporepellens, Desulfurobacterium atlanticum, Devosia pacifica, Devosia psychrophila, Devosia soli, Devosia subaequoris, Devosia submarina, Devosia yakushimensis, Dialister, Dictyoglomus thermophilum, Enterobacter, Enterobacter cloacae, Enterobacter cowanii, Enterobacteriaceae, Enterobacteriales, Escherichia, Escherichia coli, Escherichia fergusonii, Escherichia hermannii, Fimbriimonas ginsengisoli, Flavobacterium, Flavobacterium akiainvivens, Francisella novicida, Fusobacterium necrophorum, Fusobacterium nucleatum, Fusobacterium polymorphum, Haemophilus felis, Haemophilus haemolyticus, Haemophilus influenzae, Haemophilus pittmaniae, Helicobacter, Kingella kingae, Klebsiella pneumoniae, Kluyvera ascorbate, Kluyvera cryocrescens, Legionella, Legionella clemsonensis, Legionella pneumophila, Leptonema illini, Leptotrichia buccalis, Levilinea saccharolytica, Luteimonas aquatic, Luteimonas composti, Luteimonas lutimaris, Luteimonas marina, Luteimonas mephitis, Luteimonas vadose, Megamonas, Megasphaera, Meiothermus, Meiothermus timidus, Methylobacterium fujisawaense, Morax-Axenfeld diplobacilli, Moraxella, Moraxella bovis, Moraxella osloensis, Morganella morganii, Mycoplasma spumans, Neisseria cinereal, Neisseria gonorrhoeae, Neisseria meningitidis, Neisseria polysaccharea, Neisseria sicca, Nitrosomonas eutropha, Nitrosomonas halophila, Nonpathogenic organisms, OMPdb, Pectinatus, Pedobacter heparinus, Pelosinus, Propionispora, Proteobacteria, Proteus mirabilis, Proteus penneri, Pseudomonas, Pseudomonas aeruginosa, Pseudomonas luteola, Pseudoxanthomonas broeghernensis, Pseudoxanthomonas japonensis, Rickettsia rickettsia, Salinibacter ruber, Salmonella, Salmonella bongori, Salmonella enterica, Samsonia, Selenomonadales, Serratia marcescens, Shigella, Shimwellia, Solobacterium moorei, Sorangium cellulosum, Sphaerotilus natans, Sphingomonas gei, Spirochaeta, Spirochaetaceae, Sporomusa, Stenotrophomonas, Stenotrophomonas nitritireducens, Thermotoga neapolitana, Thorselliaceae, Trimeric autotransporter adhesion, Vampirococcus, Verminephrobacter, Vibrio adaptatus, Vibrio azasii, Vibrio campbellii, Vibrio cholerae, Victivallis vadensis, Vitreoscilla, Wolbachia, Yersiniaceae, Zymophilus, strains thereof, or combinations thereof.

Embodiment 8 provides the biological assembly of Embodiment 2, wherein the organelle is chosen from a mitochondria, a nucleolus, a nucleus, a ribosome, a vesicle, an endoplasmic reticulum, a Golgi apparatus, a vacuole, a lysosome, a centrosome, or a combination thereof.

Embodiment 9 provides the biological assembly of any one Embodiments 1-8, wherein the biological component has catalytic ability.

Embodiment 10 provides the biological assembly of Embodiment 9, wherein the biological component is configured to catalyze the reaction of 34-dihydroxyphenylacetic acid (DHPAC) to α-hydroxy δ-carboxymethyl cis-muconic semialdehyde, catalyze the conversion atrazine to hydroxyatrazine, or a combination thereof.

Embodiment 11 provides the biological assembly of any one of Embodiments 1-10, wherein the porous shell layer is a first porous shell layer and the assembly further comprises a second porous shell layer adjacent to the first porous shell layer.

Embodiment 12 provides the biological assembly of Embodiment 11, wherein the first porous shell layer and the second porous shell layer are held in contact through a hydrogen bond, an electrostatic interaction, an ionic bond, a covalent bond, adhesion, a physical interlocking connection, and a combination thereof.

Embodiment 13 provides the biological assembly of Embodiment 11, wherein the second porous layer comprises the same cationic component and the same anionic component as the first porous shell layer.

Embodiment 14 provides the biological assembly of Embodiment 11, wherein the second porous layer comprises a different cationic component and a different anionic component than the first porous shell layer.

Embodiment 15 provides the biological assembly of any one of Embodiments 1-14, wherein an individual porous shell layer has an average thickness in a range of from about 0.5 nm to about 50 nm.

Embodiment 16 provides the biological assembly of any one of Embodiments 1-15, wherein an individual porous shell has an average thickness in a range of from about 3 nm to about 10 nm.

Embodiment 17 provides the biological assembly of any one of Embodiments 1-16, wherein the assembly comprises from about 1 to about 30 porous shell layers.

Embodiment 18 provides the biological assembly of any one of Embodiments 1-17, wherein the assembly comprises from about 3 to about 6 porous shell layers.

Embodiment 19 provides the biological assembly of any one of Embodiments 1-18, wherein the anionic component is an acidified silane.

Embodiment 20 provides the biological assembly of Embodiment 19, wherein the acidified silane is chosen from tetramethylorthosilicate (TMOS), tetraethylorthosilicate (TEOS), tetrakis(2-hydroxyethyl)orthosilicate (THEOS), methyldiethoxysilane (MDES), 3-(glycidoxypropyl)triethoxysilane (GPMS), 3-(trimethyoxysilyl)propylacrylate (TMSPA), N-(3-triethoxysilylpropyl)pyrrole (TESPP), vinyltriethyoxysilane (VTES), methacryloxypropyltriethoxysilane (TESPM), diglycerylsilane (DGS), methyltriethoxysilane (MTMOS), trimethylmethoxysilane (TMMS), ethyltriethoxysilane (TEES), n-propyltriethoxysilane (TEPS), n-butyltriethyoxysilane (TEES), 3-aminopropyltriethoxysilane (APTS), 2-(2,4-dinitrophenylamino)propyltriethoxysilane, mercaptopropyltriethoxysilane (TEPMS), 2-(3-aminoethylamino)propyltriethoxysilane, isocyanatopropyltriethoxysilane, hydroxyl-terminated polydimethyisiloxane, triethoxysilyl-terminated polydimethylsiloxane, methyltriethoxysilane (MTES), triethoxysilyl-terminated poly(oxypropylene), nanoparticles thereof, silica nanoparticles, colloidal suspensions thereof, or a mixture thereof.

Embodiment 21 provides the biological assembly of any one of Embodiments 1-20, wherein the cationic component comprises a quaternary ammonium salt.

Embodiment 22 provides the biological assembly of Embodiments 21, wherein the quaternary ammonium salt has the structure according to formula I:

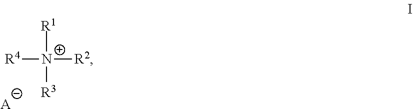

wherein

R1, R2, R3, and R4 are independently selected from the group consisting of —H, substituted or unsubstituted (C₁-C₂₀)alkyl, (C₂-20)alkenyl, (1-20)acyl, (C₄-C₂₀)cycloalkyl, (C₄-C₂₀)aryl, and combinations thereof; and

A is selected from the group consisting of F—, Cl—, Br—, and I—.

Embodiment 23 provides the biological assembly of any one of Embodiments 21 or 22, wherein the quaternary ammonium salt has the structure according to formula II:

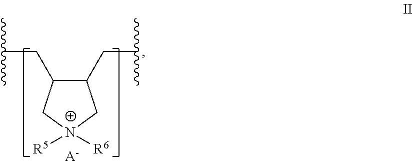

wherein

R5 and R6 are independently selected from the group consisting of —H, substituted or unsubstituted (C₁-C₂₀)alkyl, (C₂-C₂₀)alkenyl, (C₁-C₂₀)acyl, (C₄-C₂₀)cycloalkyl, (C₄-C₂₀)aryl, and combinations thereof; and

A is selected from the group consisting of F—, Cl—, Br—, and I—.

Embodiment 24 provides the biological assembly of any one of Embodiments 21-23, wherein the quaternary ammonium salt is chosen from acetylcarnitine, acetylcholine, aclidinium bromide, acriflavine, agelasine, aliquat 336, ambenonium chloride, ambutonium bromide, nnine-6plus, atracurium hesitate, behentrimonium chloride, benzalkonium chloride, benzethonium chloride, benzilone, benzododecinium bromide, benzoxonium chloride, benzyltrimethylammonium fluoride, benzyltrimethylammonium hydroxide, bephenium hydroxynaphthoate, berberine, betaine, bethanechol, bevonium, bibenzonium bromide, bis-choline tetrathiomolybdate, bretylium, bufothionine, butyrylcholine, californidine, candicine, candocuronium iodide, carbachol, carbethopendecinium bromide, carnitine, cefluprenam, cetalkonium chloride, cetrimonium, cetrimonium bromide, cetrimonium chloride, chelerythrine, chlorisondamine, chlormequat, cholamine chloride hydrochloride, choline, choline chloride, cimetropiurn bromide, cisatracurium besilate, citicoline, clidinium bromide, clofilium, cocamidopropyl betaine, cocamidopropyl hydroxysultaine, complanine, coptisine, cyanine, cyclobis(paraquat-p-phenylene), dacuronium bromide, decamethonium, 3-dehydrocarnitine, demecarium bromide, denatonium, dequalinium, didecyldimethylammonium chloride, dihydrochandonium, dimethyldioctadecylammonium chloride, dimethylphenylpiperazinium, dimethyltubocurarinium chloride, diphemanil metilsullate, diphthamide, diquat, dithiazanine iodide, domiphen bromide, doxacurium chloride, echothiophate, edelfosine, edrophonium, emepronium bromide, ethidium bromide, ethyl green, euflavine, fenpiverinium, fentonium, fluorocholine, gallamine triethiodide, gantacurium chloride, glycine betaine aldehyde, glycopyrronium bromide, guar hydroxypropyltrimonium chloride, hemicholinium-3, hexafluronium bromide, hexamethonium, hexocyclium, hydroxyethylpromethazine, hyoscine butylbromide, ipratropium bromide, isometamidium chloride, isopropamide, jatrorrhizine, laudexium metilsulfate, lucigenin, meldonium, mepenzolate, methacholine, methantheline, methiodide, methylatropine, methylhomatropine, methylnaltrexone, methylscopolamine bromide, metocurine, miltefosine, morphine methylbromide, muscarine, neurine, obidoxime, octatropine methylbromide, octenidine dihydrochloride, otilonium bromide, oxapium iodide, oxitropium bromide, oxyphenonium bromide, pahutoxin, palmatine, pancuronium bromide, pararosaniline, pentamine, penthienate, pentolinium, perifosine, phellodendrine, phosphocholine, pinaverium, pipecuronium bromide, pipenzolate bromide, poldine, polydadmac, polyquaternium, polyquaternium-7, pralidoxime, prifinium bromide, propantheline bromide, prospidium chloride, pyrvinium, quaternium-15, quinapyramine, rapacuronium bromide, rhodamine b, rimazolium, rocuronium bromide, safranin, sanguinarine, selectfluor, silane quats, sinapine, stearalkonium chloride, stercuronium iodide, succinylmonocholine, suxamethonium chloride, suxethonium chloride, tetra-n-butylammonium bromide, tetra-n-butylammonium fluoride, tetrabutylammonium, tetrabutylammonium hexafluorophosphate, tetrabutylammonium hydroxide, tetrabutylammonium tribromide, tetraethylammonium, tetraethylammonium bromide, tetraethylammonium chloride, tetraethylammonium iodide, tetramethylammonium, tetramethylammonium chloride, tetramethylammonium hydroxide, tetramethylammonium pentafluoroxenate, tetraoctylammonium bromide, tetrapropylammmium perruthenate, thiazinamium metilsulfate, thioflavin, thonzonium bromide, tibezonium iodide, tiemonium iodide, timepidium bromide, toxiferine, trazium, tridihexethyl, triethylcholine, trigonelline, trimethylglycine, trolamine salicylate, trospium chloride, tubocurarine chloride, umeclidinium bromide, vecuronium bromide, and mixtures thereof.

Embodiment 25 provides the biological assembly of any one of Embodiments 1-24, further comprising a surfactant component contacts at least a portion of one of the porous shell layers.

Embodiment 26 provides the biological assembly of Embodiment 25, wherein the surfactant is chosen from deoxycholic acid, sodium lauroyl sarcosinate, polysorbate 85 (Tween 85), and polyethylene glycol p-(1,1,3,3-tetramethylbutyl)-phenyl ether, and mixtures thereof.

Embodiment 27 provides the biological assembly of any one of Embodiments 2-26, wherein at least one of the enzyme and the protein are tethered to a granulated activated carbon.

Embodiment 28 provides the biological assembly of any one of Embodiments 2-27, wherein at least one of the enzyme, the protein, and the organelle are surrounded by a lipid bilayer.

Embodiment 29 provides the biological assembly of Embodiment 28, wherein at least one of the porous shell layers is in contact with the lipid bilayer.

Embodiment 30 provides the biological assembly of any one of Embodiments 1-29, wherein the biological structure substantially retains the ability to perform biocatalysis when the assembly is subjected to osmotic shock, lysozyme treatment, desiccation, freezing, thawing, heating, exposure to protozoa, or combinations thereof.

Embodiment 31 provides the biological assembly of Embodiment 30, wherein subjecting the biological structure to osmotic shock comprises exposing the assembly to a solution comprising ethylenediaminetetraacetic acid (EDTA) and 40% (w/v) sucrose in polybutylene.

Embodiment 32 provides the biological assembly of Embodiment 30, wherein subjecting the biological structure to lysozyme treatment comprises incubating the assembly in a mixture comprising ethylenediaminetetraacetic acid (EDTA) and lysozyme at room temperature.

Embodiment 33 provides the biological assembly of Embodiment 30, wherein, subjecting the assembly to desiccation comprises drying the assembly at a vacuum pressure of about 0.5 Pa to about 5 Pa.

Embodiment 34 provides the biological assembly of Embodiment 30, wherein freezing the assembly comprises lowering a temperature of the assembly to a temperature of about 0° C. to about −2.0° C.

Embodiment 35 provides the biological assembly of Embodiment 30, wherein heating the assembly comprises raising a temperature of the assembly to a temperature of about 30° C. to about 500° C.

Embodiment 36 provides the biological assembly of Embodiment 30, wherein the biological assembly comprises a protein an enzyme or a combination thereof, and the assembly is heated to a temperature no greater than about 5° C. lower than a denaturization temperature of the biological assembly.

Embodiment 37 provides the biological assembly of Embodiment 30, wherein exposing the biological assembly to protozoa comprises incubating the assembly with a protozoa chosen from Amoeba, Paramecium, Chilomonas, Stentor, Euglena, Volvox, or a combination thereof.

Embodiment 38 provides the biological assembly of any one of Embodiments 1-37, wherein

the biological component is a bacteria; and

the assembly includes a plurality of the porous shell layers each comprising acidified tetramethylorthosilicate (TMOS) and polydiallytdimethylammonium chloride (PDADMAC).

Embodiment 39 provides the biological assembly of any one of Embodiments 1-38, wherein

the biological component is a gram-negative bacteria;

the assembly includes a plurality of the porous shell layers each comprising acidified tetramethylorthosilicate (TMOS) and polydiallyldimethylammonium chloride (PDADMAC);

and the assembly is maintained at a temperature greater than about 25° C.

Embodiment 40 provides a method of making the biological assembly of any one of Embodiments 1-39, the method comprising:

providing or receiving the biological component; and

contacting the biological component with the anionic component followed by the cationic component to form the porous shell layer.

Embodiment 41 provides the method of making the biological assembly of Embodiment 40, wherein the porous shell layer is a first porous shell layer, and further comprising forming a second porous shell layer contacting the first porous layer.

Embodiment 42 provides the method of making the biological assembly of Embodiment 41, wherein forming the second porous shell layer comprises contacting the first porous layer with the anionic component followed by the cationic component.

Embodiment 43 provides the method of any one of Embodiments 41 or 42, further comprising removing at least a portion of the first porous layer, the second porous layer, or a combination thereof.

Embodiment 44 provides a method of using the biological assembly of any one of Embodiments 1-43 or formed according to the method of any one Embodiments 40-43, the method comprising:

contacting the biological component with a catalyzable reactant; and

catalyzing a reaction of the catalyzable reactant to a product.

Embodiment 45 provides the method of Embodiment 44, further comprising heating the assembly during the reaction.

Embodiment 46 provides the method of any one of Embodiments 44 or 45, wherein the assembly is configured to be heated to a temperature than a temperature at which a biological component that is free of the porous shell layer fails to perform canalization of the catalyzable reactant to the product.

1. A biological assembly for performing biocatalysis, the assembly comprising:
a biological component; and
a porous shell layer at least partially coating the biological component, the porous shell layer comprising an inorganic network comprising a cationic component and an anionic component.

2. The biological assembly of claim 1, wherein the biological assembly comprises at least one of a cell, an enzyme, a protein, and an organelle.

3. The biological assembly of claim wherein the cell is chosen from a eukaryotic cell, a prokaryotic cell, and a mixture thereof.

4. The biological assembly of claim 3, wherein the eukaryotic cell is chosen from a mammalian cell, yeast, or a combination thereof.

5. The biological assembly of claim 3, wherein the prokaryotic cell is a bacteria.

6. The biological assembly of claim 5, wherein the bacteria is chosen from a gram-positive bacteria, a gram-negative bacteria, and a combination thereof.

7. The biological assembly of claim 6, wherein the gram-negative bacteria is chosen from Acetic acid bacteria, Acidaminococcus, Acinetobacter baumannii, Agrobacterium tumefaciens, Akkermansia muciniphila, Anaerobiospirillum, Anaerolinea thermolimosa, Anaerolinea thermophila, Arcobacter, Arcobacter skirrowii, Armatimonas rosea, Azotobacter salinestris, Bacteroides, Bacteroides fragilis, Bacteroides ureolyticus, Bacteroidetes, Bartonella japonica, Bartonella koehlerae, Bartonella taylorii, Bdellovibrio, Brachyspira, Bradyrhizobium japonicum, Caldilinea aerophile, Cardiobacterium hominis, Chaperone-Usher fimbriae, Christensenella, Chthonomonas calidirosea, Coxiella burnetiid, Cyanobacteria, Cytophaga, Dehalogenimonas lykanthroporepellens, Desulfurobacterium atlanticum, Devosia pacifica, Devosia psychrophila, Devosia soli, Devosia subaequoris, Devosia submarine, Devosia yakushimensis, Dialister, Dictyoglomus thermophilum, Enterobacter, Enterobacter cloacae, Enterobacter cowanii, Enterobacteriaceae, Enterobacteriales, Escherichia, Escherichia coli, Escherichia fergusonii, Escherichia hermannii, Fimbriimonas ginsengisoli, Flavobacterium, Flavobacterium akiainvivens, Francisella novicida, Fusobacterium necrophorum, Fusobacterium nucleatum, Fusobacterium polymorphum, Haemophilus felis, Haemophilus haemolyticus, Haemophilus influenzae, Haemophilus pittmaniae, Helicobacter, Kingella kingae, Klebsiella pneumoniae, Kluyvera ascorbate. Kluyvera cryocrescens, Legionella, Legionella clemsonensis, Legionella pneumophila, Leptonema illini, Leptotrichia buccalis, Levilinea saccharolytica, Luteimonas aquatic, Luteimonas composti, Luteimonas lutimaris, Luteimonas marina, Luteimonas mephitis, Luteimonas vadose, Megamonas, Megasphaera, Meiothermus, Meiothermus timidus, Methylobacterium fujisawaense, Morax-Axenfeld diplobacilli, Moraxella, Moraxella bovis, Moraxella osloensis, Morganella morganii, Mycoplasma spumans, Neisseria cinereal, Neisseria gonorrhoeae, Neisseria meningitidis, Neisseria polysaccharea, Neisseria sicca, Nitrosomonas eutropha, Nitrosomonas halophila, Nonpathogenic organisms, OMPdb, Pectinatus, Pedobacter heparinus, Pelosinus, Propionispora, Proteobacteria, Proteus mirabilis, Proteus penneri, Pseudomonas, Pseudomonas aeruginosa, Pseudomonas luteola, Pseudoxanthomonas broegbernensis, Pseudoxanthomonas japonensis, Rickettsia rickettsia, Salinibacter ruber, Salmonella, Salmonella bongori, Salmonella enterica, Samsonia, Selenomonadales, Serratia marcescens, Shigella, Shimwellia, Solobacterium moorei, Sorangium cellulosum, Sphaerotilus natans, Sphingomonas gei, Spirochaeta, Spirochaetaceae, Sporomusa, Stenotrophomonas, Stenotrophomonas nitritireducens, Thermotoga neapolitana, Thorselliaceae, Trimeric autotransporter adhesion, Vampirococcus, Verminephrobacter, Vibrio adaptatus, Vibrio azasii, Vibrio campbellii, Vibrio cholerae, Victivallis vadensis, Vitreoscilla, Wolbachia, Yersiniaceae, Zymophilus, strains thereof, or combinations thereof.

8. The biological assembly of claim 1, wherein the biological component has catalytic ability.

9. The biological assembly of claim 8, wherein the biological component is configured to catalyze the reaction of 3,4-dihydroxyphenylacetic acid (DHPAC) to α-hydroxy δ-carboxymethyl cis-muconic semialdehyde, catalyze the conversion atrazine to hydroxyatrazine, or a combination thereof.

10. The biological assembly of claim 1, wherein the porous shell layer is a first porous shell layer and the assembly further comprises a second porous shell layer adjacent to the first porous shell layer.

11. The biological assembly of claim 10, wherein the first porous shell layer and the second porous shell layer are held in contact through a hydrogen bond, an electrostatic interaction, an ionic bond, a covalent bond, adhesion, a physical interlocking connection, and a combination thereof.

12. The biological assembly of claim 1, wherein an individual porous shell layer has an average thickness in a range of from about 0.5 nm to about 50 nm.

13. The biological assembly of claim 1, wherein the anionic component is an acidified silane.

14. The biological assembly of claim 13, wherein the acidified silane is chosen from tetramethylorthosilicate (TMOS), tetraethylorthosilicate (TEOS), tetrakis(2-hydroxyethyl)orthosilicate (THEOS), methyldiethoxysilane (MDES), 3-(glycidoxypropyl)triethoxysilane (GPMS), 3-(trimethyoxysilyl)propylacrylate (TMSPA), N-(3-triethoxysilylpropyl)pyrrole (TESPP), vinyltriethyoxysilane (VTES), methacryloxypropyltriethoxysilane (TESPM), diglycerylsilane (DGS), methyltriethoxysilane (MTMOS), trimethylmethoxysilane (TMMS), ethyltriethoxysilane (TEES), n-propyltriethoxysilane (TEPS), n-butyltriethyoxysilane (TEBS), 3-aminopropyltriethoxysilane (APTS), 2-(2,4-dinitrophenylamino)propyltriethoxysilane, mercaptopropyltriethoxysilane (TEPMS), 2-(3-aminoethylamino)propyltriethoxysilane, isocyanatopropyltriethoxysilane, hydroxyl-terminated polydimethylsiloxane, triethoxysilyl-terminated polydimethylsiloxane, methyltriethoxysilane (MTES), triethoxysilyl-terminated poly(oxypropylene), nanoparticles thereof, silica nanoparticles, colloidal suspensions thereof, or a mixture thereof.

15. The biological assembly of claim 1, wherein the cationic component comprises a quaternary ammonium salt.

16. The biological assembly of claim 15, wherein the quaternary ammonium salt has the structure according to formula I: 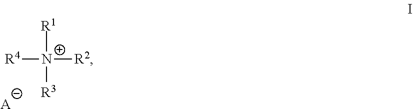
wherein
R¹, R³, and R⁴ are independently selected from the group consisting of —H, substituted or unsubstituted (C₁-C₂₀)alkyl, (C₂-C₂₀)alkenyl, (C₁-C₂₀)acyl, (C₄-C₂₀)cycloalkyl, (C₄-C₂₀)aryl, and combinations thereof; and
A is selected from the group consisting of F⁻, Cl⁻, Br⁻, and I⁻.

17. A method of making the biological assembly of claim 1, the method comprising:
providing or receiving the biological component; and
contacting the biological component with the anionic component followed by the cationic component to form the porous shell layer.

18. The method of making the biological assembly of claim 17, wherein the porous shell layer is a first porous shell layer, and further comprising forming a second porous shell layer contacting the first porous layer.

19. The method of making the biological assembly of claim 18, wherein forming the second porous shell layer comprises contacting the first porous layer with the anionic component followed by the cationic component.

20. A method of using the biological assembly of claim 1, the method comprising:
contacting the biological component with a catalyzable reactant; and
catalyzing a reaction of the catalyzable reactant to a product.
